(19) United States
(12) Patent Application Publication
(10) Pub. No.: US 2004/0010204 A1
(43) Pub. Date: Jan. 15, 2004

(54) Electronic/fiberoptic tissue differentiation instrumentation

(75) Inventors: Paul J. Weber, Fort Lauderdale, FL (US); Luiz B. Da Silva, Danville, CA (US); Kenneth B. Trauner, Sacramento, CA (US); John P. Wooldridge, Livermore, CA (US)

Correspondence Address: John P. Wooldridge, 6248 Preston Avenue, Livermore, CA, 94551, US

(73) Assignee: Pearl Technology Holdings, LLC

(21) Appl. No.: 10/401,656

(22) Filed: Mar. 28, 2003

Related U.S. Application Data
(60) Provisional application No. 60/368,353, filed on Mar. 28, 2002.

(51) Int. Cl. A61B 5/05

(52) U.S. Cl. 600/547

(57) ABSTRACT

A sensor guided needle to be used for the delivery of medication or placement of indwelling catheters, angiocatheters, spinal or epidural catheters, central lines, arterial lines, intraneoplastic, pediatric lines. The needle is comprised of an outer metal sheath with a biocompatible inner core containing sensor or signal elements. The measurements collected by the sensors are analyzed by a control unit to determine tissue type and possibly tissue state. This information can be utilized to track the progress of the needle and determine safe placement in the patient

This application claims priority to U.S. Provisional Patent Application Serial No. 60/368,353, titled “Electronic/Fiberoptic Tissue Differentiation Instrumentation” filed Mar. 28, 2002, incorporated herein by reference.

### BACKGROUND OF THE INVENTION

1. Field of the Invention

The present invention is related to the field of hypodermic needles and more specifically to hypodermic needles that are used to insert catheters or medication into the epidural space or the lumen of venous or arterial vessels.

2. Description of Related Art

Spinal epidural and caudal anesthesia was popular in the mid-1940s. However, with the advent of general anesthesia and many reports of complications such as nerve damage or spinal cord damage, the technique awaited recent re-discovery and has rightfully found great applications in pain relief for patients both during and after surgery, as well as obstetric patients and patients suffering from chronic pain.

Benefits of epidural and spinal anesthesia include allowing the patient to avoid general endotracheal intubation and its inherent risks. Use of spinal and epidural anesthetics greatly reduces the risks posed by anesthetics and the inherent elevated mortality rates for medically debilitated and fragile patients, especially those with respiratory pathology, congestive heart failure, or obesity for whom intubation is associated with high rates of complication. In addition, epidural and spinal anesthesia offers several direct medical benefits including regional hypotension for decreased surgical blood loss, reduced rates of deep venous thrombosis (DVT) and pulmonary embolism (PE) and GI surgical morbidity. Spinal anesthesia induces the gastro-intestinal tract to remain contracted or shrunken during surgery thus facilitating surgical exploration and easing the closure of abdominal wounds.

Literature suggests that the use of epidural or spinal anesthesia can reduce blood loss by as much as 25 to 50% for elective total hip replacement surgery. Additionally, blood-clotting complications may be reduced up to 50% when hip surgery is performed under lumbar epidural anesthesia. Properly performed epidural or spinal anesthesia usually helps maintain a more predictable or controllable cardio-pulmonary state during surgery as well.

Unfortunately, both spinal and epidural anesthesia application have limitations, which include a higher degree of failure than general anesthetic techniques, as well as lack of predictable duration. The greatest limitation is the failure to properly place the catheter in the epidural space, either leading to the need for a general anesthetic in a high risk patient or, inadvertent placement of a spinal anesthetic with associated comorbidities including possible respiratory suppression and postoperative spinal headaches. High cost is associated with difficulty in placing the catheter in the operating room and increased used of expensive operating room time. Failure to be able to achieve adequate spinal or epidural anesthesia may be the result of improper placement of the needle or catheter which may be the result of piercing local blood vessels or improper puncture of the various membrane levels surrounding the spinal cord. Inability to perform the needle or catheter insertion procedures accurately can cause surgical cancellations or delays.

Typically epidural anesthetic is performed following local anesthesia to the skin above the lower back where the puncture is to be made. Then a 19-gauge needle of 9 cm length is chosen for a single dose anesthesia. If continuous anesthesia is used, a 17- or 18-gauge 7.5 cm Tuohy needle is used, with a disposable plastic catheter, which receives a 23-gauge Luer-tok needle. A separate 18-gauge short bevel needle is usually used for puncturing the skin to permit the entry of the Tuohy needle. A 10 ml syringe is usually used for the “loss of resistance” test, while a 20 ml syringe may be used for the initial anesthetic injection.

Using the single dose technique for epidural anesthesia, the patient is usually placed in a lateral decubitus or flexed supine position, and a lumbar puncture is started. Unfortunately, the art of this craft is demonstrated by the need for palpation and exquisite proprioception, as well as experience on the part of the anesthesiologist. Once the needle is advanced and felt to have popped through the ligamentum flavum, a 20 ml syringe containing air or distilled water or saline is usually injected. Since the highly dense ligamentum has been pierced, the anesthesiologist usually experiences a sudden loss of resistance; this allows fluid or air to enter the peridural space.

Other methods exist to detect the epidural space, such as the “hanging drop” method of Gutierrez where a small drop of fluid is placed on the proximal hub of the needle. When the needle punctures the dura, the small drop of fluid is drawn into the needle by the negative pressure in the epidural space. Usually the anesthesiologist tries to rotate the needle in several quadrants to detect any blood or cerebrospinal fluid (CSF). The detection of blood would occur if one of the vessels were punctured, and administration of local anesthetic directly into a blood vessel could cause serious complications if significant amounts of the anesthetic were absorbed elsewhere in the body; these complications could include convulsions and cardiopulmonary arrest or shock. If the needle is poked into the subarachnoid space and CSF is obtained, then spinal anesthesia would be performed. The effects of spinal anesthesia are different from the expected effects of epidural anesthesia, and these may be unwanted in certain cases. Additionally, if CSF is obtained, that would mean that the subarachnoid space has been reached, and the likelihood of a subdural headache would be great, especially in younger patients, if large gauge needles are being used.

The major problems associated with improper placement of the needle include inadvertent spinal rather than epidural anesthetic, postural headaches, nerve damage, or respiratory paralysis and circulatory depression. Systemic reactions to local anesthetic can occur if the anesthetic was introduced into blood vessels in the epidural-peridural space, causing hypertension, loss of consciousness, and even the hazards of adrenalin in patients with arteriosclerotic heart disease. Additionally, adequate anesthesia may fail to be obtained if the catheter is placed into a peridural vein accidentally. In these cases, the onset of anesthesia may be absent or slow, and the patient may manifest an unusual circulatory reaction owing to the adrenalin injected or drowsiness, which may result eventually in convulsions.

A review of previous literature can be found in U.S. Pat. No. 6,245,044. The patent discloses a multi-element needle that can be used to more accurately position the epidural needle. U.S. Pat. Nos. 5,312,375, 5,085,631, and 5,584,820 disclose similar multi-element needle devices. However, no existing device provides the user with feedback about the type of tissue being penetrated at any instant in time.

Placement of intraluminal catheters such as intravenous lines or intraarterial lines is central to the treatment of hospitalized patients. Difficulty in placing central lines, IV lines or arterial lines can severely compromise patient care and the delay of surgical or medical procedures. For medically high-risk patients, arterial lines are required for close cardiac monitoring. Placement of lines is technically demanding. Improper placement, into the walls of the vessel lumen for example can be associated with high morbidity and may damage the vessel and compromise blood supply to the extremity it supplies. Difficulty with placement is associated with elevated costs and increased operating time for surgical procedures. The ability to visualize the levels of the arterial wall as the catheter penetrates and to sense the lumen may markedly decrease the placement failure rate.

Similarly, placement of lines in children may be extremely challenging and reduced trauma to the patient would be expected from use of a guided catheter system.

Given the limitations of current epidural needles there is a need for a device that can be used to safely and accurately guide the placement of an epidural needle or catheter. The present invention fulfills this need, and further provides related advantages.

### SUMMARY OF THE INVENTION

The object of the present invention is to provide a needle device with integrated sensors that provide the user with information about the type of tissue at the distal tip.

Another object of the present invention is to provide a device that can be used to guide needle placement in the epidural space or into the lumen of arteries or veins.

Still another object of the present invention is to provide a needle device that can be used to identify tissue planes.

These and other objects will be apparent to those skilled in the art based on the teachings herein.

In one embodiment of the present invention an epidural needle is comprised of a outer metal sheath that has a sharp distal tip optimized to puncture tissue and an inner removable core that contains optical fibers. The optical fibers are used to emit multiple wavelength light (e.g., white light source, multiple lasers or LEDs) and collect the scattered light that interacts with tissue. The spectrum of the collected light is measured with either a grating spectrometer or multiple filtered optical detectors. Software within the control electronics analyzes the spectrum and determines the type of tissue and possibly tissue state. This information is used by the user to track the progress of the needle through the various tissue layers. In normal use the control electronics can also sound an alarm when the distal tip of the needle is at the desired location or entering a sensitive tissue layer (e.g., epidural space or dura matter). The use of optical properties to distinguish tissue type and state has been documented in numerous papers. See e.g., “Tissue Optics: Applications in Medical Diagnostics and Therapy” SPIE MS102, Editor: Valery V. Tuchin, incorporated herein by reference.

In another embodiment the inner core of the needle contains a single mode fiber that can be used to perform optical coherence domain reflectometry (OCDR). This technique allows optical tissue properties to be measured ahead of the distal tip of the needle. For an example of the use of OCDR for tissue measurements refer to the paper by U. S. Sathyam, et al., Evaluation of optical coherence quantization of analytes in turbid media using two wavelengths, Applied Optics, 38(10), 2097-2104 (1999), incorporated herein by reference.

In another embodiment the inner core of the needle contains an electrical conductor that along with the outer metal sheath comprise an electrode pair that can be used to measure the electrical properties of tissue over a broad frequency range (e.g., 1 KHz-1 MHz). Software within the control electronics analyzes the measured electrical properties and determines the type of tissue and possibly tissue state. The use of electrical properties to distinguish tissue type and state has been documented in numerous papers. A good review can be found in the series of papers (all incorporated herein by reference): C. Gabriel, S. Gabriel, E. Corthout, The dielectric properties of biological tissues: I, Phys. Med. Biol. 41, 2231. S. Gabriel, R. W. Lau and C. Gabriel: The dielectric properties of biological tissues: II. Measurements in the frequency range 10 Hz to 20 GHz, Phys. Med. Biol. 41, 2251 (1996), ). S. Gabriel, R. W. Lau and C. Gabriel: The dielectric properties of biological tissues: III. Parametric models for the dielectric spectrum of tissues, Phys. Med. Biol. 41,2271 (1996).

In another embodiment the optical fibers and electrical conductor are combined within the inner core to provide a dual sensor device. The advantage of a dual sensor device is that it can provide the user with more information and greater accuracy.

These and other objects and advantages of the present invention will become apparent from the following description and accompanying drawings.

### BRIEF DESCRIPTION OF THE DRAWINGS

The accompanying drawings, which are incorporated into and form part of this disclosure, illustrate embodiments of the invention and together with the description, serve to explain the principles of the invention.

FIG. 1 shows an epidural needle in place in a patient's spine.

FIG. 2 shows a detailed cross sectional view through the center of the needle.

FIG. 3 illustrates a view of the needle inner core containing two optical fibers.

FIG. 4 shows an alternative embodiment of the inner core with a conductive wire.

FIG. 5 illustrates another embodiment utilizing a single mode optical fiber in the core.

FIG. 6 illustrates another embodiment where the optical fibers are integrated into the outer metal sheath.

FIG. 7 shows a cross sectional view through the inner core and connector element of one embodiment.

FIG. 8 shows a cross sectional view through the cable connector element for connection to the embodiment shown in FIG. 7.

FIG. 9 is a block diagram of the electronic control unit.

FIG. 10 show the measured optical signal as measured with the device in two different tissue types.

FIG. 11 shows a cross sectional view through the inner core and connector element of an embodiment that eliminates the need for a cable and external control unit.

### DETAILED DESCRIPTION OF THE INVENTION

The object of the present invention is to provide a device and method for needle placement in the epidural space or into the lumen of arteries or veins. This invention utilizes fiber optics and electrodes to determine safe placement of a needle in a patient.

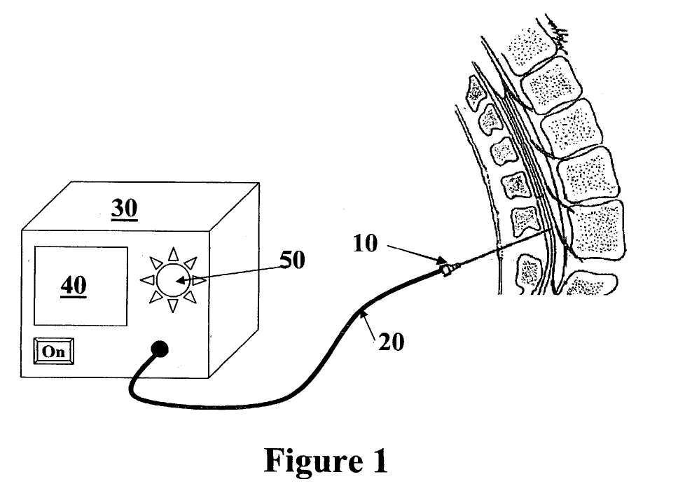

FIG. 1 illustrates how an embodiment of the present invention can be used to emplace an epidural needle. The needle 10 connects through a cable 20 to an electronic control unit 30. The control unit includes a display 40 and speaker 50 that provides the physician with information about the tissue near the tip of the needle 10. As the physician inserts the needle 10 and approaches the dura, an audible sound can be generated to warn the physician to proceed cautiously. The needle 10 with integrated sensor elements can measure optical and/or electrical properties of the tissue. The cable 20 can contain fiber optic cables and electrical cables.

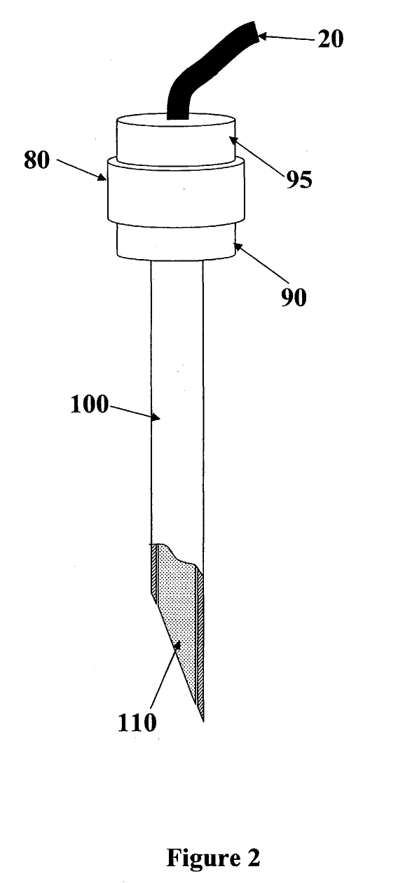

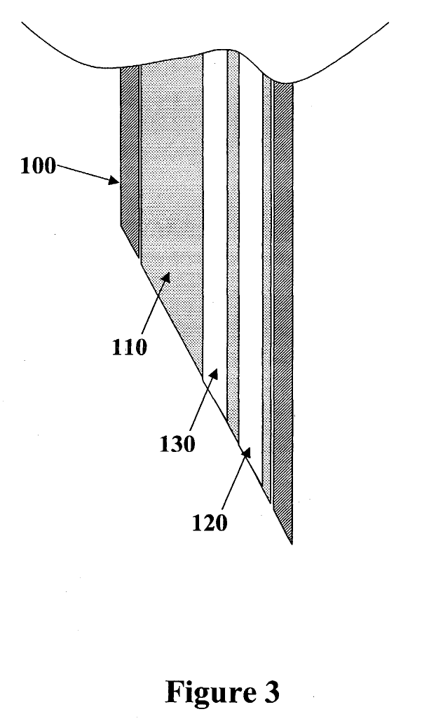

FIG. 2 shows the main section of an embodiment of the needle 10. The needle 10 comprises an outer metal sheath 100 and an internal core 110 that contains the sensor elements. The internal core 110 is integrated into connector section 80 where it also connects to cable 20. The outer metal sheath 100 is integrated into the other section of the connector 90. During use the internal core and outer metal sheath are attached by connecting sections 80 and 90 together and plugging cable 20 into connector 90. In normal use, the internal core 110 is removed after the needle 10 is placed at the desired location. In this embodiment the needle 10 including the metal sheath 100 and core 110 is a single use device and the cable 20 can be sterilized and reused (e.g., autoclaved); however, devices that may be used multiple times are within the scope of the present invention. The outer metal sheath 100 is similar to standard epidural needles (e.g., Braun, Havel's) and is manufactured using techniques commonly known in the field. The inner core 110 may be made of a biocompatible material (e.g., polyurethane, polyethylene, Teflon, glass, ceramic, and various biocompatible epoxies) or combinations thereof. Integrated into the inner core 110 are sensors or signal elements. The inner core 110 may simply be a multimode optical fiber with outside diameter closely matched to the inside diameter of the outer metal sheath to provide a snug fit FIG. 3 shows an inner core 110 that has two optical fibers integrated into it. A fiber 120 near the tip emits light and can also collect the back scattered light or fluorescent emission. A second fiber 130 collects scattered light originally emitted by the first fiber 120. The two optical fibers connect through cable 20 to the electronic control unit 30. In a simplified embodiment only one fiber 120 is used. For this embodiment the inner core can be produced by injection molding so that the optical fibers are integrated into a hard biocompatible polymer that forms the inner core 110 and the connector section 80. After molding, the distal tip is polished at an angle to match the needle tip (typically angles of less than 45 degrees relative to the needle axis).

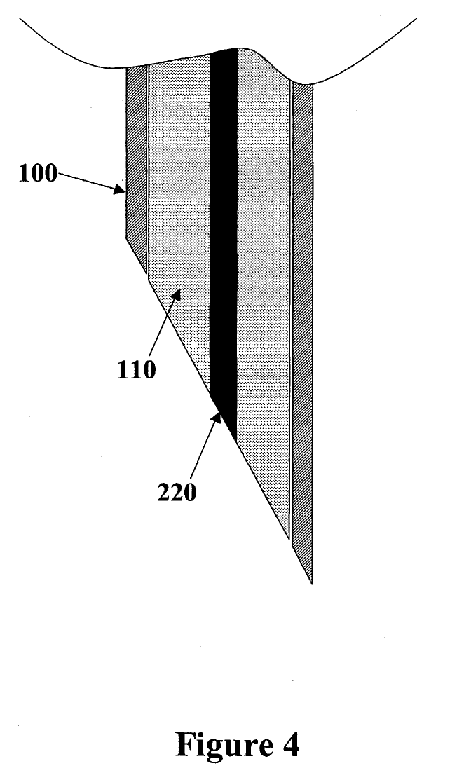

FIG. 4 shows an alternative embodiment of the inner core 110 where the center element is an electrically conductive wire 220. In this embodiment the outer metal sheath 100 acts as the second electrode and the electrical impedance between the conductive wire 220 and the metal sheath 100 is measured as a function of frequency (e.g., over the frequency range 10 kHz-10 MHz). The electrical properties of tissue are known to vary and can therefore be used to identify tissue type.

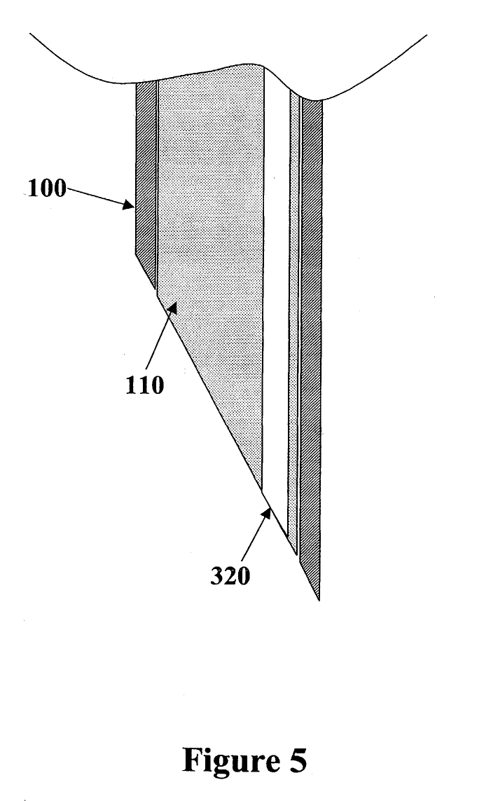

FIG. 5 shows yet another embodiment where a single mode optical fiber 320 is integrated into the core 110. The single mode optical fiber 320 is used by the electronic control unit 30 to perform optical coherence domain reflectometry (OCDR). OCDR is an optical technique that can be used to measure the optical properties of tissue along a ray extending from the fiber. OCDR can penetrate several millimeters ahead of the fiber and achieve spatial resolutions better than 10 microns. In this embodiment the electronic control unit 30 would include an OCDR module (manufactured by e.g., Optiphase, Inc. Van Nuys, Calif. USA). OCDR is known in the art. Exemplary descriptions may be found in U.S. Pat. Nos. 6,494,498 and 6,175,669, both incorporated herein by reference.

More sophisticated embodiments of this system include multiple sensor elements in the inner core 110. For example, one could combine a single mode OCDR fiber and two multimode optical fibers.

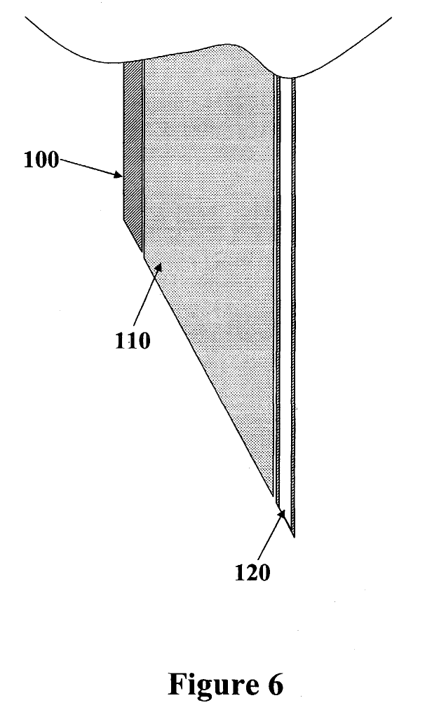

FIG. 6 shows another embodiment where the optical fiber 120 is integrated into the outer metal sheath 100. To improve sensitivity additional fiber optics can be integrated into the outer metal sheath 100. This embodiment has the advantage of eliminating the need for an inner core and places the sensing element at the distal tip. In this embodiment the metal sheath is machined to provide a slot for the optical fiber which is then bonded through a metal-glass bonding process (or with epoxy). The tip of the assembly is then polished at an angle using standard fiber optic polishing procedures to obtain a clear fiber optic surface and a sharp metal tip.

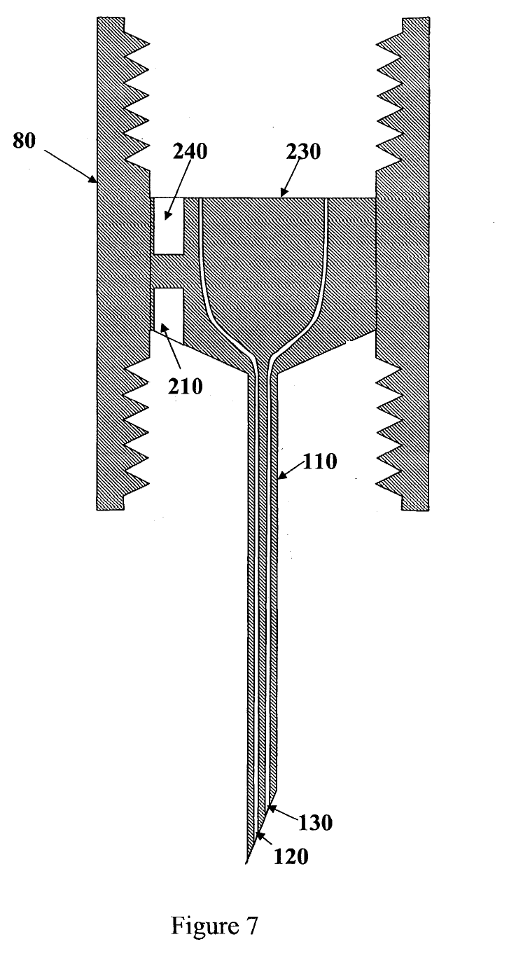

FIG. 7 shows a cross sectional view through connector 80 and the integrated inner core 110. Optical fibers 120 and 130 are integrated into the inner core 110 which has keyed holes 210 to align the outer needle sheath 100 as connector elements 90 and 80 (see FIG. 2) are screwed together. Alignment hole 210 insures that the angle polished tip of the core 110 aligns with the sharpened tip of the metal sheath 100. Alignment hole 240 insures that the cable connector element 95 attaches properly to connector element 80 to align the optical fibers. Surface 230 is optically polished to improve light coupling from the cable fiber optics to the inner core fiber optics. Although a screw type connector is shown, other connector interfaces are acceptable. For example, inner core 110 may be inserted into a standard hypodermic needle. In an alternate embodiment, optical fibers 120 and 130 may be replaced with a fiber optic bundle.

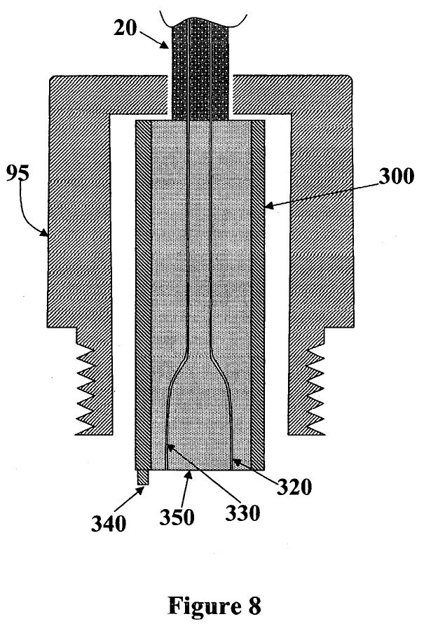

FIG. 8 shows a cross sectional view through cable connector element 95 and the internal plug assembly 300. Connector element 95 attaches to connector element 80 to deliver light from the electronic control unit 30 to the optical fibers within the inner core 110. An alignment key 340 interfaces with alignment hole 240 to insure proper fiber alignment. The plug surface 350 is optically polished to improve light coupling. In an alternative embodiment, a grin lens could be integrated into the distal end of the plug 300 to effectively transport the light to the inner core fiber optics. The use of a grin lens eliminates the need for surface 350 and 230 to be in contact (or very close) in order to effectively couple light between the fibers. By replacing the optical fibers 120 and 130 of FIG. 7 with a fiber optic bundle or a single multimode fiber optic that substantially fills the bore defined by the outer metal sheath 100, the difficulty of aligning the fibers in the plug 300 to the fibers in the core is reduced.

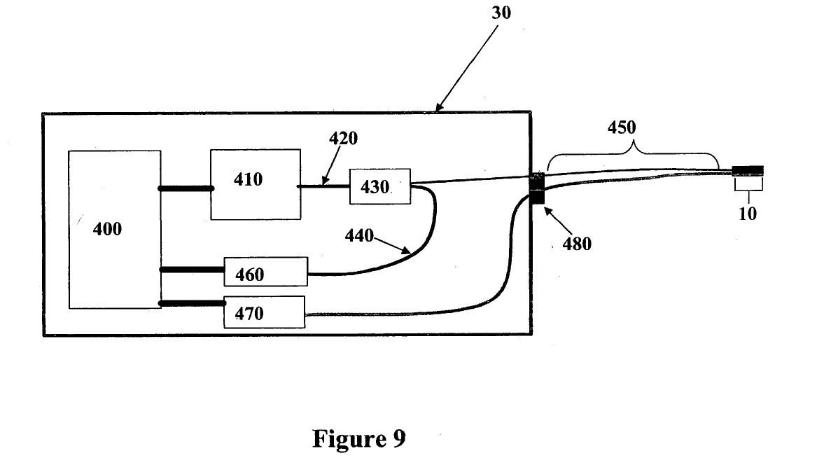

FIG. 9 shows a block diagram of the electronic control unit 30. In this embodiment the electronic control unit 30 includes an electronic control module 400, a light-generating element 410 which could be a laser or combination of lasers, a xenon light (e.g., Perkin Elmer Inc. XL100). Fiber optic cable 420 connects the light source through a splitter 430 to the connector 480. A second fiber 440 directs some of the light into a detector 460. Detector 460 is used to monitor the light being transmitted into the needle through connector 480 and the cable 450. A secondary detector 470 connects through a fiber optic to connector 480. This fiber detects the light scattered into the second fiber 130 (see FIG. 3). The two detectors 460, 470 could be grating spectrometers (e.g., Ocean Optics Inc. Dunedin Fla., USA. Model S2000) or multiple filtered diodes.

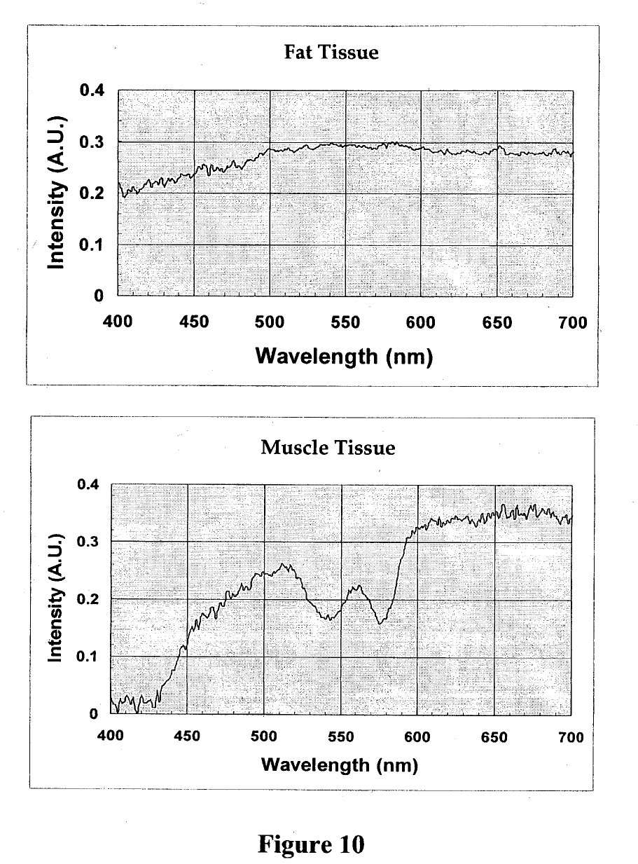

FIG. 10 is exemplary of how the spectrum of the light collected with the present invention varies depending on the tissue type and blood content. The strong absorption features near 540 nm and 570 nm are due to oxy-hemoglobin indicating the presence of blood. In normal use the control electronics monitor the measured spectrum and based on the spectral details identifies the tissue type. The user is notified when the needle is at the desired tissue. In one embodiment the control electronics have a table of spectra for all the possible different tissue types that may be measured. During use the analysis software identifies the spectra that best matches the measured spectra and provide a diagnosis.

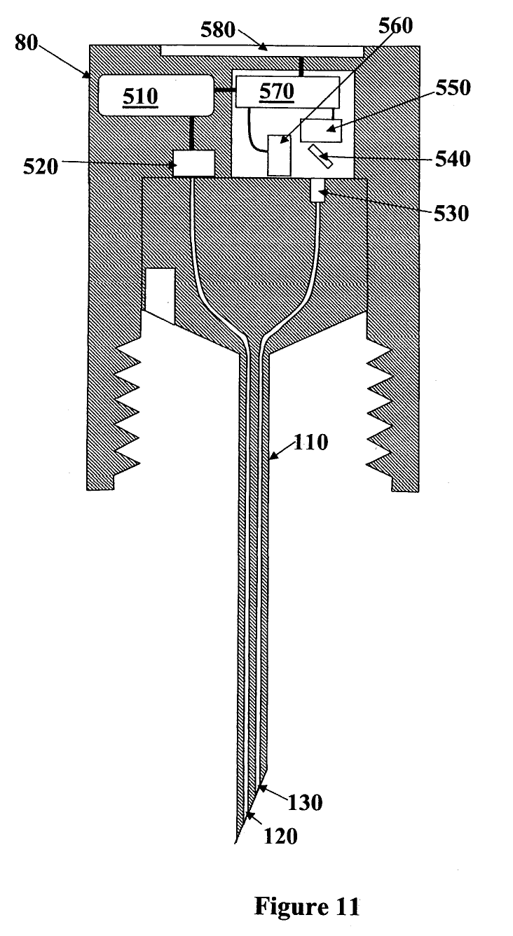

FIG. 11 shows a cross-sectional view through an alternative embodiment where the light source and filtered optical detectors are integrated into the inner core connector (80, FIG. 2) along with a battery and necessary electronics. In this embodiment, light generated by one or multiple LEDs 520 is proximity coupled into an optical fiber 120. Light collected by optical fiber 130 is transported through a grin lens 530 to a dielectric mirror 540. At the dielectric mirror 540 part of the light is reflected and couples into photodiode detector 560. The light transmitted through dielectric mirror 540 couples into photodiode detector 550 that is filtered to detect a different part of the optical spectrum. By using addition mirrors and filters it would be possible to have additional spectral measurements. The signals from the photodiode are processes by an electronic module 570 and relevant information diplayed on an LCD display 580 or alternatively a group of coded LEDs. This embodiment eliminates the need for a cable and external control unit. By using a white light LED or multiple wavelength LEDs, in combination with filtered optical detectors, it is possible to identify a variety of tissue types.

The above descriptions and illustrations are only by way of example and are not to be taken as limiting the invention in any manner. One skilled in the art can substitute known equivalents for the structures and means described. The full scope and definition of the invention, therefore, is set forth in the following claims.

We claim:

1. An apparatus, comprising:
a hypodermic needle having an outer sheath that has a sharp distal tip for puncturing tissue, wherein said outer sheath defines a hollow inner bore; and
an inner removable core within said hollow inner bore, wherein said core includes a sensor element for inclusion in a system that provides a user with information about the type of tissue at said distal tip.

2. The apparatus of claim 1, wherein said sensor element comprises at least one optical fiber.

3. The apparatus of claim 1, wherein said sensor element comprises a single mode optical fiber.

4. The apparatus of claim 3, further comprising an optical coherence domain reflectometry system connected to said single mode fiber.

5. The apparatus of claim 1, wherein said outer sheath comprises an electrically conductive material, wherein said sensor element comprises an electrical conductor that along with said outer metal sheath comprise an electrode pair for inclusion in said system, wherein said system measures electrical properties of tissue.

6. The apparatus of claim 5, further comprising means for providing current to said electrode pair.

7. The apparatus of claim 6, wherein said means for providing current is capable of providing current within a frequency range of about 1 KHz to about 1 MHz.

8. The apparatus of claim 1, wherein said outer sheath comprises an electrically conductive material, wherein said sensor element comprises at least one optical fiber and at least one electrical conductor within said inner removable core to provide a dual sensor device.

9. The apparatus of claim 1, further comprising an optical fiber integrated into said outer sheath.

10. The apparatus of claim 1, further comprising an electronic control unit operatively attached to said sensor element to analyze data collected from said sensor element.

11. The apparatus of claim 10, wherein said electronic control unit comprises:
means for providing energy to tissue at said distal tip; and
means for analyzing the interaction of said energy with said tissue.

12. The apparatus of claim 1I, wherein said energy is selected from the group consisting of electromagnetic energy and optical energy.

13. The apparatus of claim 11, wherein said means for analyzing the interaction of said energy with said tissue is selected from the group consisting of a grating spectrometer and a multiple filtered optical detector.

14. The apparatus of claim 1, wherein said hypodermic needle is an epidural needle.

15. The apparatus of claim 11, wherein said means for analyzing the interaction of said energy with said tissue comprises software for analyzing the interaction of said energy with said tissue.

16. The apparatus of claim 10, further comprising an alarm that can sound when said distal tip is at a desired location or entering a particular tissue layer.

17. The apparatus of claim 1, wherein said core comprises a biocompatible material.

18. The apparatus of claim 1, wherein said core comprises material selected from the group consisting of polyurethane, polyethylene, glass, ceramic and epoxy.

19. The apparatus of claim 1, further comprising a plurality of optical fibers integrated into said outer sheath.

20. The apparatus of claim 1, wherein said sensor element comprises a plurality of optical fibers.

21. The apparatus of claim 1, further comprising a first connector attached to said core, wherein said first connector comprises means to attach said inner core to said needle.

22. The apparatus of claim 1, wherein said inner removable core comprises a fiber optic bundle.

23. The apparatus of claim 1, wherein said inner removable core comprises a multimode fiber optic.

24. The apparatus of claim 1, further comprising at least one fiber optic embedded within said core, wherein said fiber optic comprises a first index of refraction, wherein said core comprises a second index of refraction, wherein said first index of refraction is larger than said second index of refraction.

25. The apparatus of claim 21, further comprising a second connector comprising means for attachment to said first connector, wherein said second connector is operatively attached to a cable having means for transmitting a signal from said sensor element to means for analyzing said signal.

26. The apparatus of claim 1, further comprising a connector for attachment to said core, wherein said connector comprises means for providing energy to said sensor element, means for receiving and analyzing a signal generated by said sensor element, and means for providing analyzed signal information to a user.

27. An apparatus, comprising:
an removable core for placement within the hollow inner bore of a hypodermic needle, wherein said core includes a sensor element for inclusion in a system that provides a user with information about the type of tissue at said distal tip.

28. An apparatus, comprising:
a hypodermic needle having an outer sheath that has a sharp distal tip for puncturing tissue; and
at least one optical fiber integrated into said outer metal sheath.

29. A method, comprising:
inserting a sensor guided needle into tissue;
collecting data from said sensor guided needle, wherein said data is indicative of tissue type;
analyzing said data to determine tissue type; and
providing said analyzed data of tissue type to a user.

30. The method of claim 29, wherein said sensor guided needle comprises a hypodermic needle having an outer sheath that has a sharp distal tip for puncturing tissue, wherein said outer sheath defines a hollow inner bore and an inner removable core within said hollow inner bore, wherein said core includes a sensor element for inclusion in a system that provides a user with information about the type of tissue at said distal tip.

31. The method of claim 30, wherein said sensor element comprises at, least one optical fiber.

32. The method of claim 30, wherein said sensor element comprises a single mode optical fiber.

33. The method of claim 32, further comprising an optical coherence domain reflectometry system connected to said single mode fiber.

34. The method of claim 30, wherein said outer sheath comprises an electrically conductive material, wherein said sensor element comprises an electrical conductor that along with said outer metal sheath comprise an electrode pair for inclusion in said system, wherein said system measures electrical properties of tissue.

35. The method of claim 30, wherein said outer sheath comprises an electrically conductive material, wherein said sensor element comprises at least one optical fiber and at least one electrical conductor within said inner removable core to provide a dual sensor device.

36. The method of claim 30, further comprising an optical fiber integrated into said outer sheath.

37. The method of claim 30, further comprising an electronic control unit operatively attached to said sensor element to analyze data collected from said sensor element.

38. The method of claim 30, wherein said sensor element comprises at least one fiber optic embedded within said core, wherein said fiber optic comprises a first index of refraction, wherein said core comprises a second index of refraction, wherein said first index of refraction is larger than said second index of refraction.

39. The method of claim 29, wherein said sensor guided needle comprises a hypodermic needle having an outer sheath that has a sharp distal tip for puncturing tissue; and at least one optical fiber integrated into said outer metal sheath.
